(19) United States
(12) Patent Application Publication
(10) Pub. No.: US 2013/0079402 A1
(43) Pub. Date: Mar. 28, 2013

(54) Use of at Least One Dialkyl Thiosulfonate or Thiosulfinate for Reducing the Number of Apicomplexa in an Animal

(71) Applicants: David Bravo, Yverdon-Les-Bains (CH); Hyun Lillehoj, West Friendship, MD (US)

(72) Inventors: David Bravo, Yverdon-Les-Bains (CH); Hyun Lillehoj, West Friendship, MD (US)

(73) Assignee: Pancosma S.A., Geneva (CH)

(21) Appl. No.: 13/637,241

(22) PCT Filed: Apr. 1, 2011
(86) PCT No.: PCT/CH11/00069

§ 371 Date: Dec. 4, 2012

(30) Foreign Application Priority Data
Apr. 1, 2010 (FR) 1001375

(51) Int. Cl. A61K 31/255

(52) U.S. Cl. CPC A61K 31/255
USPC 514/517

(57) ABSTRACT

The invention relates to the use of at least one dialkyl thiosulfonate or thiosulfinate in order to reduce the number of apicomplexa, such as coccidioides and, in particular, Eimeria, in monogastric animals, such as poultry or pigs, or ruminants, such as cattle, sheep or goats.

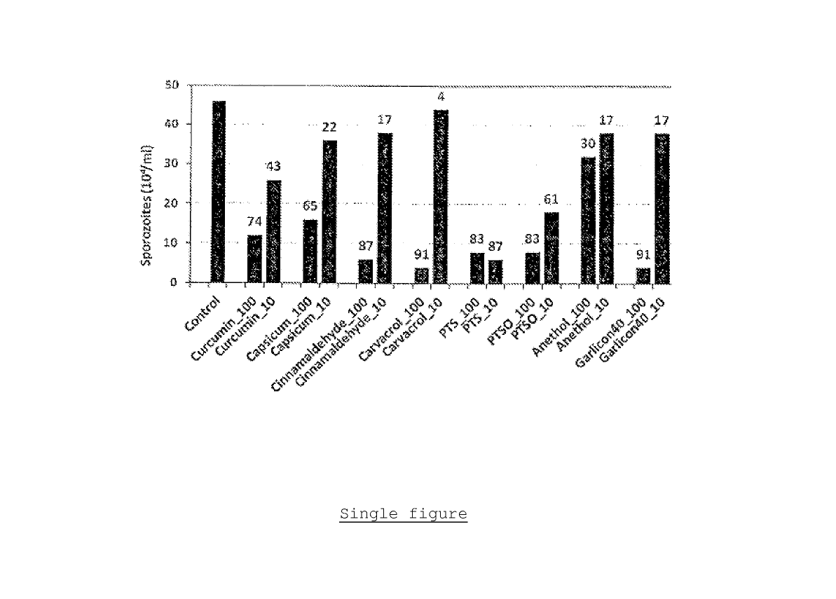

The invention relates to the use of at least one dialkyl thiosulfinate or thiosulfonate for reducing the number of apicomplexa present in monogastric animals, such as poultry or pigs, or ruminants, such as cattle, sheep or goats.

### BACKGROUND OF THE INVENTION

Apicomplexa or sporozoa are single-cell organisms which are parasites of vertebrates or of invertebrates. These parasites can cause various pathological conditions.

Thus, for example, avian coccidiosis, which is a universal intestinal protozoan infection that seriously disturbs the feeding and growth of poultry, is caused by several parasites of the apicomplexa group, in particular of the Eimeria genus, which infest the intestine and propagate from one animal to another by ingestion of infected oocysts.

Neospora and cryptosporidia, other organisms belonging to the apicomplexa group, can also infest the animal intestine.

All these infections lead to economic losses for the farming industry and, at the current time, there is no effective vaccine against them.

The conventional strategies for controlling infections of this type are very expensive since they are based mainly on chemoprophylaxis. Furthermore, the continual emergence of drug-resistant strains, such as Eimeria strains, combined with the hardening of the regulations and banning of the use of drugs against coccidia in industrial poultry farming, mean that new approaches and strategies for disease control are required.

Because of the complexity of the host's immunity and of the lifecycle of the parasites, it proves to be particularly difficult to develop new strategies for intervention or new vaccines against apicomplexa.

The current tendency is therefore to use vaccines based on live parasites to control coccidiosis. At least ten vaccines of this type are commercially available worldwide. All these vaccines have recourse to infected live oocysts in order to produce a real infection which is intended to trigger a natural immunization that protects chickens aged from 3 to 4 weeks, which corresponds to the age at which the majority of cases of coccidiosis break out.

This vaccination technique has the major drawback that live oocysts can be produced only by live chickens, which requires a lot of work and involves numerous manipulations.

Document US 2006/122266 mentions the use of, among other compounds, allicin, which is a diallyl thiosulfinate, i.e. a dialkylene thiosulfinate, and therefore a product containing 2 unsaturations, for treating malaria.

Document EP 2 110 128 cites propyl propylthiosulfinate (PTS) and propyl propylthiosulfonate (PTSO) as antibacterial and antimicrobial compounds; all the organisms cited in these documents are bacteria, i.e. prokaryotes, whereas the apicomplexa are eukaryotes; the cell structures between prokaryotes and eukaryotes are totally different.

Document WO 2004/084645 relates to the uses of allicin. No use against apicomplexa is mentioned or suggested in this document.

The extract from the WPI databank, Week 199838, Thomson Scientific, London, GB, AN 1998-440169 & HU 9 602 190 A2 of May 28, 1998, mentions the use of a combination of garlic oil, thymol, peppermint oil, citrus oil and diluents or supports for treating and preventing coccidiosis; given that there is a large variety of constituents in these compounds, nothing in this document makes it possible to say which of these components is active, alone or in combination with other constituents.

The publication by M. R. Lewis et al., “Effects of dietary inclusion of plant extracts on the growth performance of male broiler chickens”, British Poultry Science, Longman Group, GB LNKD-DOI: 10.1080/00071660301940, vol. 44, No. Suppl. 1, Jan. 1, 2003, page S43, XP008104094 ISSN: 0007-1668, and also the publication by E. Demir et al., “The use of natural feed additives as alternatives for an antibiotic growth promoter in broiler diets”, British Poultry Science, Longman Group, GB LNKD-DOI: 10.1080/00071660301944, vol. 44, No. Suppl. 1, Jan. 1, 2003, pages S44 and S45, XP008104095 ISSN: 0007-1668, relate to the growth of animals which are made to ingest various compounds, including garlic; no effect on apicomplexa is mentioned in these documents.

Document US 2009/018194 relates to the use of PTS or of PTSO for treating plants.

Document EP 1 721 534 relates to the use of derivatives of the Allium genus as preservatives in the food and food-processing industry.

### SUMMARIZING DESCRIPTION OF THE INVENTION

The major objective of the invention is to combat the apicomplexa present in the body of monogastric animals, such as poultry or pigs, or of ruminants, such as cattle, sheep or goats.

The research carried out by the inventors has enabled them to find agents capable of destroying apicomplexa. Surprisingly, these agents are known compounds, dialkyl thiosulfinates and thiosulfonates, in particular di(n-propyl) thiosulfinate and di(n-propyl) thiosulfonate, which will be referred to, respectively, as PTS and PTSO in the remainder of the description.

The use of these compounds has proved to be particularly effective for destroying apicomplexa, in particular Eimeria.

In addition, these compounds can be produced independently of the animal for which they are intended, i.e. without requiring oocysts, from natural products or by organic synthesis.

Moreover, they have the surprising advantage of being effective even at very low doses.

Other characteristics and advantages of the invention will now be described in detail in the description which follows and which is given with reference to the appended single figure which is a comparative diagram representing the reduction in number of sporozoites as a function of the compounds used.

### DETAILED DESCRIPTION OF THE INVENTION

The dialkyl thiosulfinates and thiosulfonates are compounds corresponding to the following formula F:

F−SOₐ−S−R′

in which:
R and R′, which may be identical or different,
each represent an alkyl group; and
a is 1 or 2.

Preferably, the alkyl groups R and R′ comprise from 1 to 5 carbon atoms.

Furthermore, propyl groups, generally n-propyl groups, are more particularly used as R and R′ groups.

Within one and the same compound of formula F, the R and R′ groups are preferably identical.

For a dialkyl thiosulfinate, in formula F, a is 1.

For a dialkyl thiosulfonate, a is 2.

It is advantageous to use a dipropyl thiosulfinate and/or a dipropyl thiosulfonate, in particular PTS and/or PTSO, as compound(s) of formula F.

Indeed, although the PTS may be a synthetic product, it can advantageously be extracted from plants belonging to the family Alliaceae (according to the APG phylogenetic classification) and in particular to the Allium genus, which is a member of this family.

The most well-known plants belonging to the Allium genus are leek (Allium ampeloprasum), onion (Allium cepa L.), cultivated garlic (Allium sativum L.), shallot (Allium ascalonicum), Welsh onion (Allium fistulosum L.) and chive (Allium schoenoprasum L.).

According to the invention, an extract of garlic, in particular of cultivated garlic (Allium sativum L.) is preferably used. It is generally an extract of garlic bulb.

The PTSO can be obtained by synthesis or decomposition (or oxidation) of PTS.

The obtaining of PTS by extraction and of PTSO is in particular described in patent application EP-A1-1 721 534, from page 15, line 34 to page 16, line 26.

The thiosulfinates can also be obtained according to the process described in patent application FR-A-2 813 884.

Of course, it is possible to use mixtures in any proportions of
several dialkyl thiosulfinates;
several dialkyl thiosulfonates;
at least one dialkyl thiosulfinate and at least one dialkyl thiosulfonate.

Studies have shown that animals, in particular poultry, do not develop resistance against these compounds, thereby making their use in industrial farming particularly advantageous.

The PTS and/or the PTSO are used prophylactically.

For their use prophylactically, the dialkyl thiosulfinate(s) and/or thiosulfonate(s) can be added to the feed for the animals, for example, by mixing them daily with their ration.

The amount used is then generally from 2 mg per kg to 60 mg per kg of feed dry matter.

They can optionally be diluted in the water given to the animals, in a proportion from 1 to 30 mg/l of water, on the basis of the average amount drunk by the animal.

The invention applies to monogastric animals such as poultry or pigs, or ruminants, such as cattle, sheep or goats.

### Comparative Example

The antiparasitic properties of the following compounds were tested:
a capsicum oleoresin (Capsicum frutescens pigment) containing 6% by weight of a mixture of capsaicin and dihydrocapsaicin;
carvacrol;
cinnamaldehyde;
curcumin;
anethole;
PTS (according to the invention);
PTSO (according to the invention); and
an extract (according to the invention) called “Garlicon 40” containing 400 ppm of organosulfur compounds characteristic of garlic/onion, 6.7% by weight of PTS and 34.3% by weight of PTSO.

A first series of samples consisting of an aqueous solution containing 100 ppm of the abovementioned test compounds, and a second series of samples containing only 10 ppm of these compounds, were prepared.

Next, in order to be able to test the effect of the abovementioned compounds on Eimeria, the following was carried out. Freshly sporulated oocysts were obtained from Eimeria tenella and were stored at 4° C. in a 2.5% (weight/volume) potassium dichromate solution until use.

The oocysts were then sterilized with bleach before isolating sporozoites. The walls of the oocysts were mechanically destroyed for 5 to 7 seconds using a ball mill having balls of 0.5 mm.

The product obtained was then incubated for 45 minutes at 41° C. in a PBS buffer solution containing 0.014 mol/l of taurodeoxycholic acid and 0.25% by weight of trypsin in order to release the sporozoites.

The fluids released by the excystation were filtered and the sporozoites collected were washed three times with RPMI-1640 medium with stirring at 2100 revolutions per minute for 10 minutes at 4° C.

Solutions of 10⁶ sporozoites per milliliter were then prepared, and were left to incubate with the same volume of sample of test compound for 24 hours.

The viability of the sporozoites was then measured according to the trypan blue exclusion method.

The results can be seen on the appended single figure, in which the numbers located above each column indicate the percentage inhibition by the corresponding compound and the numbers which follow the name of the compound indicate its concentration.

A measurement was also carried out with a control solution (free of test compound) called “Control” on the figure.

It appears, firstly, that all the compounds tested at a concentration of 100 ppm, except anethole, have a marked inhibitory or destructive effect on the sporozoites of Eimeria tenella (of at least 61%) and, secondly, that curcumin, cinnamaldehyde, carvacrol, PTS and PTSO or the Garlicon 40 extract are the most effective (at least 74%).

Furthermore, it is noted with surprise that only PTS and PTSO show a high effectiveness, respectively of 87% and 61%, at a concentration level of only 10 ppm.

What is more, surprisingly, PTSO even has a higher effectiveness at 10 ppm than at 100 ppm.

1. The prophylactic use of at least one compound corresponding to the following formula F:
R−SOₐ−S−R′
in which:
R and R′, which may be identical or different, each represent an alkyl group; and
a is 1 or 2;
for reducing the number of apicomplexa in a monogastric animal or a ruminant.

2. The use as claimed in claim 1, in which, in formula F, the R and R′ groups are alkyl groups comprising from 1 to 5 carbon atoms.

3. The use as claimed in claim 2, in which, in formula F, the R and R′ groups are propyl groups.

4. The use as claimed in claim 1, in which the R and R′ groups are identical.

5. The use as claimed in claim 1, in which, in formula F, a is 1.

6. The use as claimed in claim 1, in which the compound of formula F is dipropyl thiosulfinate.

7. The use as claimed in claim 1, in which, in formula F, a is 2.

8. The use as claimed in claim 1, in which the compound of formula F is dipropyl thiosulfonate.

9. The use as claimed in claim 1, in which the compound(s) of formula F is (are) at least in part in the form of an extract of a plant belonging to the family Alliaceae.

10. The use as claimed in claim 9, in which the compound(s) of formula F is (are) at least in part in the form of an extract of a plant belonging to the Allium genus.

11. The use as claimed in claim 10, in which the plant of the Allium genus is cultivated garlic (Allium sativum L.).

12. The use as claimed in claim 1, in which the animal is a monogastric animal.

13. The use as claimed in claim 12, in which the animal is a fowl.

14. The use as claimed in claim 1, in which the animal is a ruminant.

15. The use as claimed in claim 14, in which the animal is a bovine.

16. The use as claimed in claim 1, in which the apicomplexa are coccidia.

17. The use as claimed in claim 16, in which the coccidia are Eimeria.

18. The use as claimed in claim 1, in which the apicomplexa are Neospora or Cryptosporidia.

19. The use as claimed in claim 1, in a proportion of from 2 to 60 mg/kg of feed dry matter or from 1 to 30 mg/l of drinking water.
